(19) United States
(12) Patent Application Publication
(10) Pub. No.: US 2004/0219233 A1
(43) Pub. Date: Nov. 4, 2004

(54) Composition comprising the extract of cistanche deserticola Y.C. MA showing enhancing activity of the neurite outgrowth and neurotrophic effects

(75) Inventors: Sun-Yeou Kim, Seoul (KR); Jin-Young Hur, Seoul (KR); Sun-Kyoung Jeong, EuiWang (KR)

Correspondence Address: FOLEY AND LARDNER, SUITE 500, 3000 K STREET NW, WASHINGTON, DC, 20007, US

(73) Assignee: KYUNG HEE UNIVERSITY

(21) Appl. No.: 10/854,203

(22) Filed: May 27, 2004

Related U.S. Application Data
(63) Continuation of application No. PCT/KR03/01622, filed on Aug. 12, 2003.

(30) Foreign Application Priority Data
Aug. 12, 2002 (KR) 10-2002-0047412

(51) Int. Cl. A61K 35/78

(52) U.S. Cl. 424/725

(57) ABSTRACT

The present invention relates to a composition comprising an extract of Cistanche deserticola Y. C. having nerve growth factor (NGF) similar activity for the prevention and treatment of neuronal degenerative brain disease.

The extracts from Cistanche deserticola have potent neuronal cell protective activity by promoting neurite outgrowth and acting as a NGF therefore it can be used as the therapeutics or health food for treating and preventing neuro-degenerative brain diseases.

### CROSS REFERENCE TO RELATED APPLICATION

This application is a continuation patent application of PCT Patent Application No. PCT/KR03/1622, which was filed on Aug. 12, 2004, designating the United Stares of America, now abandoned.

### FIELD OF THE INVENTION

The present invention relates to an extract of Cistanche deserticola Y. C. having nerve growth factor (NGF) similar activity and a composition comprising the same having preventing and treating degenerative brain disease.

### BACKGROUND OF THE INVENTION

In the twentieth century, as the average life span of human has been increasing with the rapid development of life science and medicine, new social problems including increased population ratio of older people are coming to the front, especially, the geriatric neuronal diseases such as stroke, Alzheimer's disease (AD), Parkinson's disease (PD) etc., which are fatal functional disorder of neuronal system, have been increased.

The growth, differentiation and death of neuronal cell in neuronal system are important control processes in general development, the establishment of tissue specific function and the maintenance of homeostasis respectively.

The death of neuronal cells is classified into two types, apoptosis and necrosis: Neuronal necrosis is caused by the injuries induced by the rapid unbalance of intracellular ion concentration, the expansion of cytoplasm and mitochondria and the cell lyses of nuclear membrane after the lysis of cytoplasm, which means the acute cell death caused by the sudden physical or chemical injury such as ischemic anemia, hypothermia, stroke and so on (Tomei et al.; Proc. Nat'l. Acad. Sci. U.S.A., 90, pp853-857, 1993). It is not affected by protein synthesis inhibitors and its representative example occurred in neuronal system is caused by the injury of ionic glutamic acid receptor activation. Apoptosis is called as programmed cell death and is followed by the characteristic morphological changes comprising the membrane change such as cell shrinkage, membrane blebbing and the breakdown of cellular skeleton, and the nuclear change such as chromatin condensation and the like (Kerr J. F., J Pathol., 107(3), pp217-219, 1972: Arends M. J. & Morris R. G, Am. J. Pathol., 136(30), pp 593-608, 1990), which makes the cell form an apoptotic body, a small structure surrounded by a membrane, together with the loss of mitochondrial function. The formed apoptotic body is completely removed by the phagocytosis of neighboring cell or macrophage without inflammatory response induced by the exudation of cytoplasm contents.

All the behaviors in multi-cellular organism such as cell growth, differentiation and migration etc, are controlled by the controlling factors existing outside of cells. Neuronal cell also requires such controlling factors, which includes all proteins released from target cell affecting the neuronal cell growth, differentiation and survival in CNS (Central Nervous System) and PNS (Peripheral Nervous System). There are five involving factors: NGF (nerve growth factor), BDNF (Brain-derived neurotrophic factor), NT-3 (Neurotrophin-3), NT-4 and NT-5, which is different from each other in respect to the origin of reproduction, the differentiation and the expression appearance, and the targeting position, however, similar to the arrangement of consisting amino acid sequences between species. Neurotrophic factor inhibits apoptosis in neuronal system. The apoptosis occurring when neurotrophic factor is in deficiency, is one of the cell death dependent to most of new protein synthesis and cell death-involved genes. It has been well identified that neurotrophic factors inhibit the predetermined death of neuronal cell in the development of neuronal system.

NGF, one of above neurotropic factors (NFs) having those functions has been extracted and isolated from snake venom or mouse sarcoma by Levi-Montalcini in the early 1950's, and it has been reported that the neurite growth of chic sympathetic ganglia neuron is promoted by several folds with incubating in the above NF extract (Levi-Montalcini R., Birth Defects Orig. Artic. Ser., 19(4), pp3-22, 1983).

The above NGF has been reported to promote neuronal differentiation till now, however, it is newly found that it inhibits neuronal degenerative death resulting in preventing from the numerical decrease of neuron recently. The receptors of NGFs are present in afferent sensory neuronal ganglia, brain and sympathetic nerve-governing organ. It has been reported that NGFs are bio-synthesized in sympathetic nerve-governing organ such as heart in vivo, absorbed at terminal end of neuron, transferred from axon in reverse direction to neuronal cell and NGFs promotes protein synthesis (Mahalik T. J., Investig. Dermatol. Symp. Proc., August; 2(1), ppl4-18, 1997).

There are many reports on the protective function of NGF from neuronal cell injury in CNS: for example, cholinergic axotomy of Fimbria-fornix prevents from cholinergic addition from cholinergic neuronal cell of forebrain base to hypothalamus, which makes cholinergic neuronal cell be degenerative slowly and if NGF is added thereto after axotomy, the degeneration of cholinergic neuronal cell is completely inhibited. If high concentration of BDNF is added, the similar effect to that of NGF can be obtained from cholinergic axotomy (Hefti F. J., Neurosci., August;6(8), pp2155-2162, 1986). Regarding on the correlation-between NGF and peripheral nerve, the role of NGF in matured animal has not been completely identified, however, it has been reported that if the antibody against NGF is injected, the breakdown of sympathetic ganglia occurs and the activities of norepinephrine synthetase such as tyrosine hydroxylase and dopamine beta-hydroxylase are also reduced (Levi-Montalcinin et al.;. Bull. Soc. Sci. Med. Grand Duche Luxemb., 115(2), pp69-74, 1978).

NGF may play an important role in neuron regeneration process after neuronal injury on the base of the report which if NGF was injected outside the cell, the number of survived NGF-sensitive cells and the governing degree of neuron for corresponding organ were increased and the developmental change was weakened (Zettler C. et al; Brain Res., 538(2), pp251-262, 1991). Those results showed that the governing degree of NGF in organ is closely correlated with that of sympathetic neuron.

It has been reported that about 50% of developing neurons in normal developing process in neuronal system are removed by the apoptosis and NF excreted from target cells, determines neuronal survival (Barres B. A. et al; Development, 118(1), pp283-95, 1993).

NFs such as NGFs is essentially required for neuronal cell to survive, grow and differentiate in normal status. However, those NGFs could not penetrate BBB (Blood Brain Barrier) because of their high molecular weight. Therefore, they could not show satisfactory therapeutic effect to treat degenerative brain disease. NGF synthesis should be further induced and further, the development of their substitutes having similar role to NGF has been urgently required now.

Cistanche deserticola Y.C. MA belonged to Orobanchaceae, is distributed in the area of alkaline earth, dried river and sandy region. It has been used as materials of Chinese medicine as a restorative and reported to comprise several enzymes, fatty lipid, trace alkaloid and crystalline neutral substances (Chung B. S. and Shin M. K.; HyangyakDaesacheon, Youngrimsa, pp888-889, 1998).

It has been several reports that cistanoside, cistanoside F, tubuloside A, tubuloside B, 2′-acetylacetoside, echinacoside, 3′-alpha-rhamnopyranoside, isoaceteoside, acetoside, syringalide A component were isolated from Cistanche deserticola Y.C. MA (Wang Y M, et al.; Yao Xue Xue Bao, 35(11), pp839-842, 2000)

However, there has been not reported or disclosed about therapeutic effect for brain disease of Cistanche deserticola Y.C. MA in any of above cited literatures, the disclosures of which are incorporated herein by reference.

To investigate an effect of Cistanche deserticola Y.C. MA on neuronal growth and differentiation through several biochemical experiments and to confirm whether the crude extract and non-polar solvent soluble extract play an important role in inhibiting neuronal cell apoptosis, main cause of degenerative brain diseases, and in promoting the NGF induction or not, the inventors of the present invention have intensively carried out several molecular biological experiments togethering with micro-observation through cell line culture, and finally completed present invention by confirming that the crude extract and non-polar solvent soluble extract inhibit the neuronal apoptosis, promote the generation of NGFs and show neuronal cell protective activity.

These and other objects of the present invention will become apparent from the detailed disclosure of the present invention provided hereinafter.

### SUMMARY OF THE INVENTION

Accordingly, it is an object of the present invention to provide a pharmaceutical composition comprising a crude extract of Cistanche deserticola Y.C. MA as an active ingredient in an effective amount to treat and prevent degenerative brain disease by protecting neuronal cell.

The present invention also provides a use of above extract for the preparation of pharmaceutical composition to treat and prevent degenerative brain disease by protecting neuronal cell in mammal or human.

The present invention also provides a health food or food additives comprising above extract for the prevention or alleviation of degenerative brain disease by protecting neuronal cell.

### BRIEF DESCRIPTION OF THE DRAWINGS

The above and other objects, features and other advantages of the present invention will more clearly understood from the following detailed description taken in conjunction with the accompanying drawings, in which;

FIG. 1 shows photographs of neurite growth of PC12 cell line treated with inventive extracts or NGF in microscope with magnification ×200;

FIG. 2 shows the effect of neurite growth treated with inventive extracts or NGF;

FIG. 3 shows the neurite outgrowth treated with various concentrations of NGF in microscope with magnification ×200;

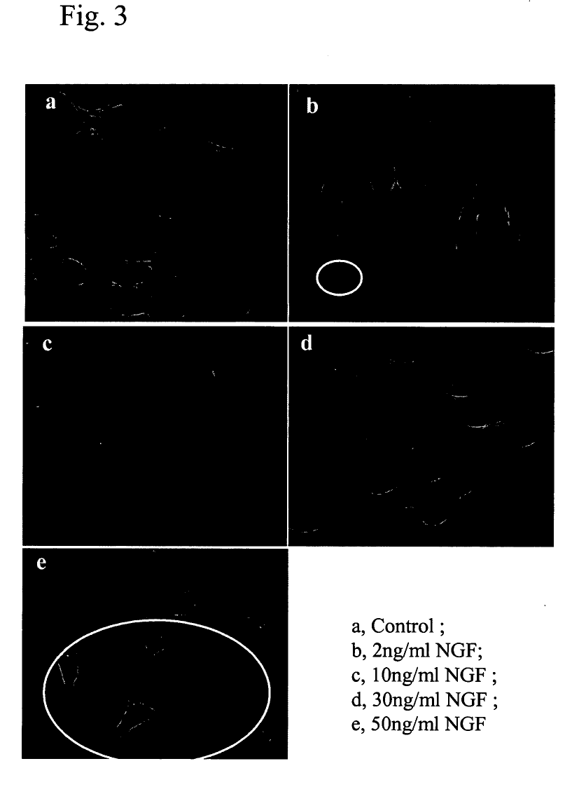

FIG. 4 represents the effect of NGF on neurite growth;

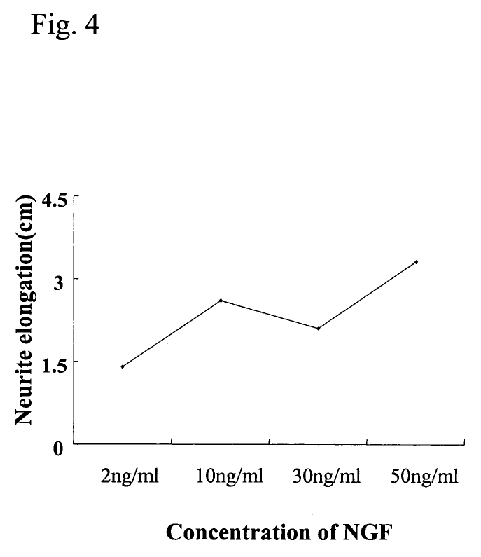

FIG. 5 represents the cell viability of 10 μg/ml of ethyl acetate soluble extract-treated cell observed by LDH and MTT assay;

FIG. 6 presents the DNA fragmentation in 10 μg/ml of ethyl acetate soluble extract-treated cell;

FIG. 7a is the picture of 50 ng/ml of NGF treated cell; FIG. 7b is the picture of 50 ng/ml of NGF treated & withdrawn cell; FIG. 7c is the picture of 10 μg/ml of ethyl acetate soluble extract-treated & withdrawn cell; FIG. 7d is the picture of serum-deprived and 10 μg/ml of ethyl acetate soluble extract-treated cell;

FIG. 8a depicts the NGF gene expression observed by electrophoresis; FIG. 8b depicts the graph representing the expressed level;

FIG. 9 depicts immunocytochemical assay of NGF receptor expression in PC12 cell;

FIG. 10 depicts the effect of ethyl acetate extract of the present invention on passive recognition when administered to scopolamine-induced amnesia mouse.

### DETAILED DESCRIPTION OF THE INVENTION

Accordingly, it is an object of the present invention to provide a pharmaceutical composition comprising the crude extract, polar solvent soluble or non-polar solvent soluble extract of Cistanche deserticola Y.C. MA as an active ingredients for the treatment and prevention of degenerative brain disease by protecting neuronal cell.

Above described crude extract comprises the extract prepared by extracting plant material with water, lower alcohols such as methanol, ethanol, preferably methanol and the like, or the mixtures thereof.

Above described polar solvent soluble extract can be prepared by extracting above crude extract with polar solvent, for example, water, lower alcohol such as methanol, ethanol, preferably butanol and the like, or the mixtures thereof.

Above described non-polar solvent soluble extract can be prepared by extracting above crude extract with non-polar solvent, for example, hexane, ethyl acetate or dichloromethane, preferably ethyl acetate.

It is an object of the present invention to provide a use of a crude extract, polar solvent soluble or non-polar solvent soluble extract of Cistanche deserticola Y.C. MA for the preparation of therapeutic agent for the treatment and prevention of degenerative brain disease by protecting neuronal cell in human or mammal.

It is an object of the present invention to provide a method of treating or preventing degenerative brain disease by protecting neuronal cell in a mammal comprising administering to said mammal an effective amount of crude extract, polar solvent soluble or non-polar solvent soluble extract of Cistanche deserticola Y.C. MA, together with a pharmaceutically acceptable carrier thereof.

It is another object of the present invention to provide a health food or food additives comprising above extract, together with a sitologically acceptable additive for the prevention and improvement of degenerative brain disease by protecting neuronal cell.

Above described degenerative brain disease comprises stroke, Alzheimer's disease (AD), Parkinson's disease (PD), senile dementia and the like.

Above described crude extract may be extracted from any of Cistanche genus plants such as Cistanche deserticola, C. salsa or C. ambigua.

The pharmaceutical composition of the present invention can contain about 0.01˜50% by weight of the above extract based on the total weight of the composition.

The health food of the present invention comprises above extracts as 0.01 to 80%, preferably 1 to 50% by weight based on the total weight of the composition.

Above health food can be contained in health food, health beverage etc, and may be used as powder, granule, tablet, chewing tablet, capsule, beverage etc.

An inventive extracts isolated from Cistanche deserticola Y.C. MA may be prepared in accordance with the following preferred embodiment.

Hereinafter, the present invention is described in detail.

An inventive extract of Cistanche deserticola Y.C. MA can be prepared in detail by following procedures,

The inventive crude extract of Cistanche deserticola Y.C. MA can be prepared by follows; Cistanche deserticola Y.C. MA is dried, cut, crushed and mixed with 5 to 25-fold, preferably, approximately 10 fold volume of distilled water, lower alcohols such as methanol, ethanol, butanol and the like, or the mixtures thereof, preferably methanol; the solution is treated with hot water at the temperature ranging from 20 to 100° C., preferably from 60 to 100° C., for the period ranging from 1 to 24 hours with extraction method by the extraction with hot water, cold water, reflux extraction, or ultra-sonication extraction with 1 to 5 times, preferably 2 to 3 times, consecutively; the residue is filtered to obtain the supernatant to be concentrated with rotary evaporator, at the temperature ranging from 20 to 100° C., preferably from 50 to 70° C. and then dried by vacuum freeze-drying, hot air-drying or spray drying to obtain dried crude extract powder of Cistanche deserticola Y.C. MA which can be soluble in water, lower alcohols, or the mixtures thereof.

Additionally, polar solvent soluble and non-polar solvent soluble extract of present invention can be prepared by following procedure; the crude extract prepared by above step, is suspended in water, and then is mixed with 1 to 100-fold, preferably, 1 to 5-fold volume of non polar solvent such as ethyl acetate, chloroform, hexane and the like; the non-polar solvent soluble layer is collected to obtain non-polar solvent soluble extract of the present invention and remaining polar solvent soluble layer is collected to obtain polar solvent soluble extract of the present invention which is soluble in water, lower alcohols, or the mixtures thereof. Also, above described procedures may be modified or subjected to further step to fractionate or isolate more potent fractions or compounds by conventional procedure well-known in the art, for example, the procedure disclosed in the literature (Harbome J. B. Phytochemical methods: A guide to modern techniques of plant analysis, 3^{rd} Ed. pp6-7, 1998).

To investigate the effect of Cistanche deserticola Y.C. MA on neuronal growth and differentiation through several biochemical experiments and to confirm whether the crude extract and non-polar solvent soluble extract play an important role in inhibiting neuronal cell apoptosis, main cause of degenerative brain diseases, and in promoting the NGF induction or not, together with micro-observation through cell line culture, and then it is confirmed that the crude extract, polar solvent soluble and non-polar solvent soluble extract inhibit the neuronal apoptosis, promotes the generation of NFs and shows neuronal cell protective activity.

In accordance with another aspect of the present invention, there is provided a pharmaceutical composition comprising the crude extract, polar solvent soluble or non-polar solvent soluble extract of Cistanche deserticola Y.C. MA prepared by above preparation method for the treatment and prevention of degenerative brain disease by protecting neuronal cell as active ingredients.

It is another of the present invention to provide a treating method and preventing method comprising administering a pharmaceutical composition comprising said extract prepared by above preparation method to degenerative brain of mammals including human.

The inventive composition for treating and preventing degenerative brain disease by protecting neuronal cell may comprises above extracts as 0.01˜50% by weight based on the total weight of the composition.

The inventive composition may additionally comprise conventional carrier, adjuvants or diluents in accordance with a using method well known in the art. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to them. For topical administration, the extract of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to administer at the amount ranging 10 g/kg, preferably, 1 to 3 g/kg by weight/day of the inventive extract or compounds of the present invention. The dose may be administered in single or divided into several times per day. In terms of composition, the amount of inventive extract should be present between 0.01 to 50% by weight, preferably 0.5 to 40% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intracutaneous, intrathecal, epidural or intracerebroventricular injection.

Also, the present invention provide a composition of the health food beverage for the prevention and improvement of degenerative brain disease by protecting neuronal cell adding above described extracts 0.01 to 80% by weight, amino acids 0.001 to 5% by weight, vitamins 0.001 to 2% by weight, sugars 0.001 to 20% by weight, organic acids 0.001 to 10% by weight, sweetener and flavors of proper amount.

Above described extract of Cistanche deserticola Y.C. MA can be added to food and beverage for the prevention and improvement of degenerative brain disease by protecting neuronal cell.

To develop for health food, examples of addable food comprising above extracts of the present invention are various food, beverage, gum, vitamin complex, health improving food and the like, and can be used as power, granule, tablet, chewing tablet, capsule or beverage etc.

Also, the extract of the present invention will be able to prevent, and improve allergic disease and non-allergic inflammation disease by comprising to child and infant food, such as modified milk powder, modified milk powder for growth period, modified food for growth period.

Above described composition therein can be added to food, additive or beverage, wherein, the amount of above described extract in food or beverage may generally range from about 0.1 to 80 w/w %, preferably 1 to 50 w/w % of total weight of food for the health food composition and 1 to 30 g, preferably 3 to 10 g on the ratio of 100 ml of the health beverage composition.

Providing that the health beverage composition of present invention contains above described extract as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract therein are various food, beverage, gum, vitamin complex, health improving food and the like.

The inventive composition may additionally comprise one or more than one of organic acid, such as citric acid, fumaric acid, adipic acid, lactic acid, malic acid; phosphate, such as phosphate, sodium phosphate, potassium phosphate, acid pyrophosphate, polyphosphate; natural anti-oxidants, such as polyphenol, catechin, α-tocopherol, rosemary extract, vitamin C, green tea extract, licorice root extract, chitosan, tannic acid, phytic acid etc.

The above extract of Cistanche deserticola Y.C. MA may be 20 to 90% high concentrated liquid, power, or granule type.

Similarly, the above extract of Cistanche deserticola Y.C. MA can comprise additionally one or more than one of lactose, casein, dextrose, glucose, sucrose and sorbitol.

Inventive extract of the present invention have no toxicity and adverse effect therefore; they can be used with safe.

It will be apparent to those skilled in the art that various modifications and variations can be made in the compositions, use and preparations of the present invention without departing from the spirit or scope of the invention.

### BEST MODE FOR CARRING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### EXAMPLES

The following Reference Example, Examples and Experimental Examples are intended to further illustrate the present invention without limiting its scope.

### Example 1

### Preparation of the Crude Extract of Cistanche deserticola

1 kg of dried Cistanche deserticola purchased from Kyung-dong Market located in Seoul was cut into small pieces, mixed with 1.5 L of 85% methanol (water:methanol=15:85) and the mixture was stayed still at room temperature for 1 day.

And the mixture was subjected to sonication extraction (Branson Co. U.S.A.) for 1 hour twice and the extract was filtered with filter paper to remove the debris.

The filtrates were pooled and concentrated by rotary evaporator (N-1000, Eyela Co. Japan) at 55˜65° C. under reduced pressure and dried with freezing dryer (Speed Spec 3000, Bio-Rad Co. U.S.A.) to obtain 455 g of dried crude extract. The dried powder was dissolved in distilled water (100 mg/ml).

### Example 2

### Preparation of Polar Solvent and Non-polar Solvent Soluble Extract

The dried extract prepared in Example 1 was subject to fractionation by following procedure.

2-1. Preparation of Ethyl Acetate Soluble Fraction

300 ml of distilled water was added to 455 g of the crude extract obtained in Example 1. 3000 ml of chloroform was added thereto in separatory funnel, shaken vigorously to divide into ethyl acetate soluble layer and water soluble layer.

Above water soluble layer was mixed with equivalent amount of ethyl acetate and then divided into ethyl acetate soluble layer and water soluble layer. The fractionation process was repeated 4-5 times.

Above ethyl acetate soluble layer was concentrated by rotary evaporator, dried with freeze dryer to obtain 8.2 g of ethyl acetate soluble extract.

2-2. Preparation of Butanol/Water Soluble Fraction

Water soluble layer was fractionated by mixing with butanol and finally, 11.83 g of butanol soluble extract and 26 g of water soluble extract were obtained to use as a sample in the following experiments.

### Reference Example 1

### Preparation of Samples

The crude extract and ethyl acetate soluble extract prepared in Example 1 and 2 were suspended in PBS(pH 7.2) at the concentration of 1.0 mg/ml respectively and the each suspension was filtered with 0.2 μm membrane filter (Milipore Co., Ltd.) to remove bacteria. In case that sample was not dissolved completely, the sample was resuspended in DMSO (Sigma Co., Ltd) as a final concentration of >0.1% and filtered to sterilize as described above manner.

### Reference Example 2

### Cell Culture

3×10⁴ PC12 cells were grown on lOOmm diameter culture dish (TPP Co., Ltd., Switzerland) in DMEM (Gibco BRL Co., Ltd., USA), supplemented with 2.0 g/liter sodium bicarbonate (NaHCO₃), 5% horse serum, 10% fetal bovine serum which was inactivated at 55° C. for 30 mins before use and 1% penicillin-streptomycin antibiotics (1000 U/ml), at 37° C. in 5% CO₂ and 95% air condition in a humidified incubator.

Used medium was changed with 10 ml fresh DMEM 4 times per week and cells were subcultured 3-4 times per week.

### Reference Example 3

### Treatement with Samples

Cells (1×10⁷ cells/well) on 10 mm culture dish were treated with 500 μl of trypsin-EDTA (Gibco BRL Co., Ltd., U.S.A.) to separate from the dish and 10 ml of fresh medium was added thereto so as to neutralize trypsin-EDTA solution and obtain cell suspension.

For cell counting, 0.4% of trypan blue diluted in phosphate buffered saline was added to 20 μl of the cell suspension and the number of living cells was counted by cell counter.

Cell adhesion enzyme, 50 μg/ml of poly-D-lysine (Sigma Chemical Co., St. Louis, Mo., U.S.A.) diluted with PBS buffer was aliquoted by 2 ml into 6-well cell culture plate, incubated at 37° C. for 1 hour and washed with PBS (pH 7.2). 6-well plate was dried and used in following experiment.

The cells (1×10⁵ cells/well) prepared by above procedure were seeded on 6-well plate and incubated for 24 hours. Cells were treated with the crude extract of Cistanche deserticola in Example 1(10 μg/ml) or NGF(50 ng/ml) and incubated at 37° C. in 5% CO₂ and 95% air condition in a humidified incubator for 6 days with changing medium every 2 days.

### Experimental Example 1

### Physiological Activity of the Extract of Cistanche deserticola

To assess the physiological activity of the extract of Cistanche deserticola, 10 μg/ml the inventive extract of Example 1 or Example 2 was treated to PC12 cells (Korea Cell Line Bank, the Cancer Research Institute of the Seoul National University College of Medicine).

Various concentrations of NGF were treated to the cells for determining the most effective concentration of NGF and thereby selected NGF was used as a control to verify neurite outgrowth.

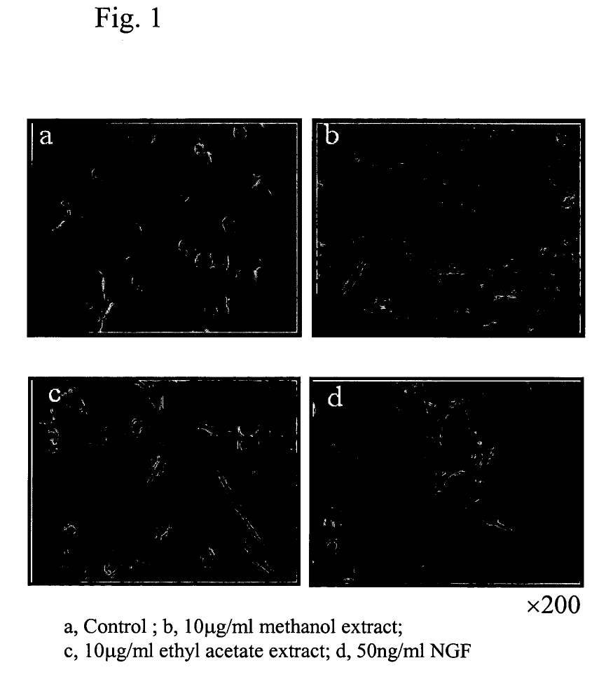

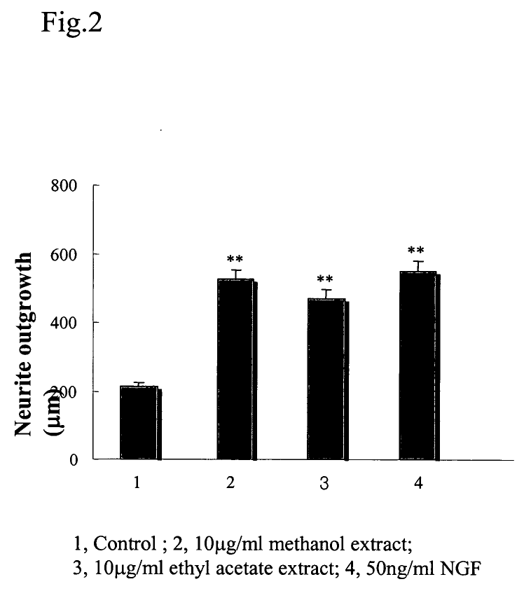

After 2 days, it was started to form neurite and on 6^{th} day, neurite outgrowth was occurred to differentiate to neurofilament. And ethyl acetate soluble extract prepared in Example 2 showed the highest effect on growth of neurite (FIG. 1a, FIG. 1b, FIG. 1c, FIG. 1d and FIG. 2).

### Experimental Example 2

### Effect of Cistanche deserticola Fractions on Neurite Outgrowth

10 μg/ml of each extract was treated to PC12 cell for 24 hours. After 24 hours incubation, the medium was changed with fresh media containing 50 ng/ml of NGF, 1% FBS and 2% HS, and neurites of nerve cell body were observed and photographed using by an phase-contrast microscope(model CK-25, Olympus Co., U.S.A.) at regular interval everyday. The length of neurite was measured on the photography.

Neurite extension was evaluated as 0 in case that the neurite was not seen, as 1 in case that the neurite length was equivalent to one diameter of the cell body, as 2 in case that the neurite length was 2 times longer than the diameter of the cell body and as 3 in case that neurite length was over 3 times longer (FIG. 1a, FIG. 1b, FIG. 1c, FIG. 1d and FIG. 2). In control group, 50 ng/ml NGF was treated therewith. All data are expressed as the mean ±S.D. The evaluation of statistical significance was determined by student's-T test (**p<0.01, FIG. 2 and Table 2).

Inventive Cistanche deserticola extract showed the excellent effect on neurite outgrowth (*p<0.01).

TABLE 2

| Sample | Ratio of neurite (% of control) |
|---|---|
| Control | 100% |
| NGF | 133 ± 0.21% |
| Cistanche deserticola | 132 ± 0.27% |
| crude extract | |

### Experimental Example 3

### LDH Release Assay of Cistanche deserticola Extract

The LDH (lactate dehydrogenase) inhibitory effect of inventive extract was investigated to measure the extent of apoptosis by them.

In order to determine LDH released into media in NGF-free condition, PC12 cells were treated with ethyl acetate soluble extract in Example 2.

As experimental groups, PC12 cells were also treated with 10 μg/ml of ethyl acetate soluble extract in Example 2 for 6 days and cultured with fresh medium for additional 24 hours, and 30 μl of culture medium was transferred to 96-well plate.

To carry out LDH release assay (Kim et al., J. Neurosci. Res., 53, pp426-432, 1998), medium and 30 μl of 1 mg/ml NADH (dissolved in 0.75 mM sodium pyruvate) in 96-well plate was incubated at 37° C. for 30 mins followed by adding the coloring reagent and the mixture was incubated further at 37° C. for 20 mins. After quencing the reaction by adding 0.4N sodium hydroxide to reaction mixture, the UV absorbance of above solutions was measured at 405 nm.

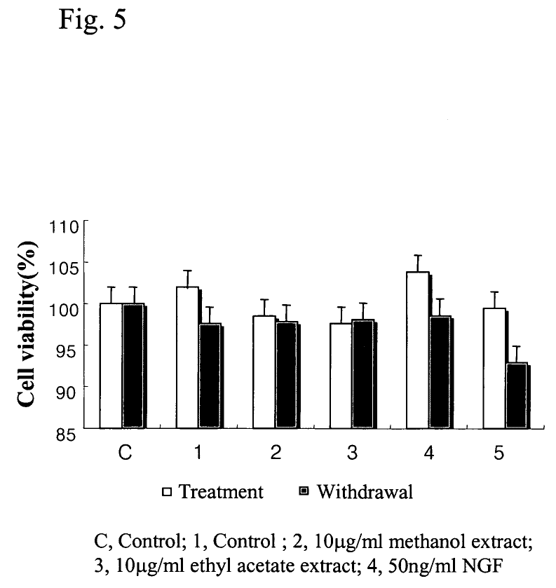

As shown in FIG. 5, the LDH level of ethyl acetate soluble extract-withdrawn group was decreased due to apoptosis.

### Experimental Example 4

### MTT Assay

To investigate the apoptotic effect of Cistanche deserticola extract was determined by (3-[4,5-dimethylthiazol-2-yl]-2,5-diphenyl tetrazolium bromide (MTT) assay method.

PC12 cells (2×10⁴ cells/well) were seeded in 96-well plate under NGF-free condition and after 24 hours incubation, the cells were treated with 10 μg/ml of ethyl acetate soluble extract of Cistanche deserticola for another 48 hours. After 2 days the medium was discarded and 150 μl of MTT solution (0.05 mg/ml suspended in medium, Sigma Co.) was added to the cell and reacted at 37° C. 4 hours later, MTT was removed and 150 μl of DMSO was dropped into each well to dissolve crystals. At 570 nm, UV absorbance was measured by microplate reader (ELISA reader, Molecular devices Co., U.S.A.) to calculate the cell viability.

As shown in FIG. 5, the result demonstrates that cell viability was declined and the apoptosis was remarkably increased in Cistanche deserticola extract-treated group.

### Experimental Example 5

### Apoptosis Assay of Cistanche deserticola Extract

To test whether apoptosis occurred or not when inventive ethyl acetate soluble extract was removed, DNA fragmentation assay was performed.

PC12 cells (2.5-5×10⁶ cells/well) were grown on 100 mm diameter culture dish in DMEM medium for 24 hours, and the medium was aspirated and the cells was washed with PBS. The fresh medium containing sample was added thereto and cells were incubated at 37° C. in 5% CO₂ and 95% air condition in a humidified incubator for 6 days. Media was changed with fresh DMEM media containing inventive extract every 2 days.

Cells were harvested by adding 0.25% Trypsin-EDTA, centrifuged at the speed of 1000 rpm for 5 mins to remove the supernatant thereof. Cell precipitates were resuspended in lysis buffer (5 mmol/liter Tris-HCl (pH 7.4), 5 mmol/liter EDTA, 0.5% Triton X-100) using 1.5 ml eppendorf tube and after 15 mins the mixture was centrifuged at 12,00 rpm for 20 min. The resulting supernatant was transferred to new tube, mixed with 1.0 μl of RNase and incubated at 37° C. for 1 hour. After incubation, 1 μl of proteinase and SDS(final conc. 1%) was added thereto and the reaction mixture was incubated at 50° C. for another 2 hours. The mixture was subjected to phenol-chloroform extraction. Phenol-chloroform solvent mixture equivalent amount to the above mixture was put into the tube, vortexed for 15 seconds and centrifuged at 12,000 rpm for 10 mins. The supernatant in a new tube was mixed with 1/10 volume of 3M sodium acetate and 2 volumes of absolute ethanol, and incubated at R. T. for 30 mins followed by centrifugation with the speed of 12,000 rpm at the temperature of 4° C. for 10 mins. At that time, the supernatant was discarded and the pellet was dried completely to be suspended in 20 μl of TAE buffer.

To investigate the above-purified DNA fragments, the electrophoresis was carried out by using agarose (Gibco BRL Co., U.S.A.) gel and 1×TAE buffer.

10 μl of purified DNA mixed with staining solution was aliquoted, loaded in the agarose gel and subjected to electrophoresis at 100 volts for 40 minutes. DNA fragmentation was measured using Gel-DOC (Model Gel DOC 2000, Bio-Rad, U.S.A.).

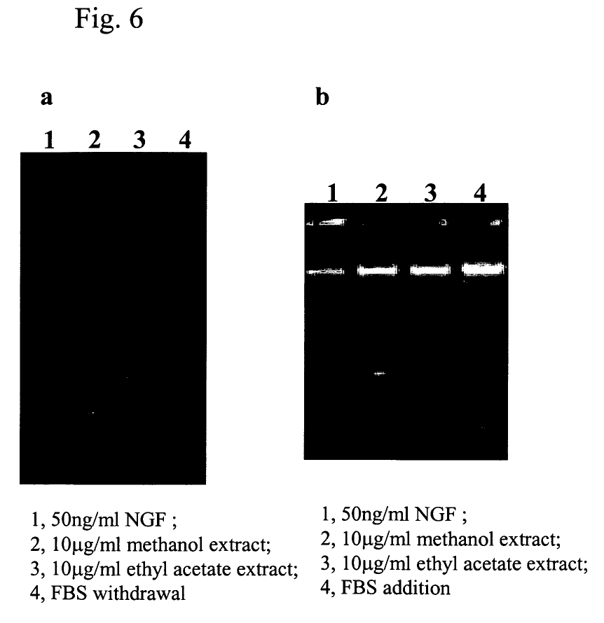

At the result of FIG. 6a, lane 1 is 50 ng/μl of NGF treated group, lane 2 is 10 μg/ml of the crude extract of Cistanche deserticola-treated group, lane 3 is 10 μg/ml of the ethyl acetate soluble extract of Cistanche deserticola-treated group and lane 4 is the FBS-deprivation group. In the cell treated with inventive crude extract and ethyl acetate soluble extract and removed, DNA fragmentation was detected, which meant that the cell apoptosis occurred.

FIG. 6b shows fragmented DNAs isolated from inventive extract-treated cells after serum deprivation resulting in apoptosis. Lane 1 is 50 ng/μl of NGF treated group, lane 2 is 10 μg/ml of the crude extract-treated group, lane 3 is 10 μg/ml of the ethyl acetate soluble extract-treated group and lane 4 is the FBS-treated group. When the crude extract and ethyl acetate soluble extract was treated to the cells, DNA fragmentation was not occurred.

As the inventive extract was treated after serum deprivation, cellular DNA was not fragmented and thereby it was confirmed that the inventive extract inhibited apoptosis of the cell.

### Experimental Example 6

### Effect of Cistanche deserticola on Apoptosis of the Cell Using Fluorescence

To determine the effect of Cistanche deserticola on apoptosis of the cell, FACS analysis was performed in this experiment.

PC12 cell was grown in complete medium for 24 hours. 24 hours later, medium was changed with serum-depriving medium containing 2% horse serum and 1% FBS and NGF or ethyl acetate soluble-extract prepared in Example 2-1 was added thereto. Another 24 hours incubation was further subjected. Cell suspension was centrifuged at 200×g for 5 mins and the supernatant was discarded. Cell pellet was suspended in 100 μl of annexin V-FITC solution dissolved in the buffer containing 10 mM HEPES(pH 7.4), 150 mM sodium chloride, 5 mM potassium chloride, 1 mM magnesium chloride and 1.8 mM calcium chloride and incubated at room temperature for 5 mins in the dark. At that time, 100 μl of HEPES buffer was dropped into FACS microtube and 20 μl of propidium iodide(PI, 100 μg/ml in HEPES buffer) was added thereto.

PI, a kind of fluorescence dye, can be bound to DNA of the cell and determine the amount of DNA-bound for respective cells, if apoptosis happens.

Eventually, FACS analysis was performed in order to quantify the extent of apoptosis induction.

If there are lots of living cells, the dots representing the living cell are located on the left bottom of the graph. The dots representing apoptotic cells are located on right bottom of the graph and the dots representing necrotic cells are located on right top of the graph. When apoptosis occurs, dots are moved to left bottom of the resulting graph.

In case that ethyl acetate soluble extract is removed from the media, it is found that dots representing cell are moved onto the right bottom of graph as the time goes by, which means apoptosis occurs.

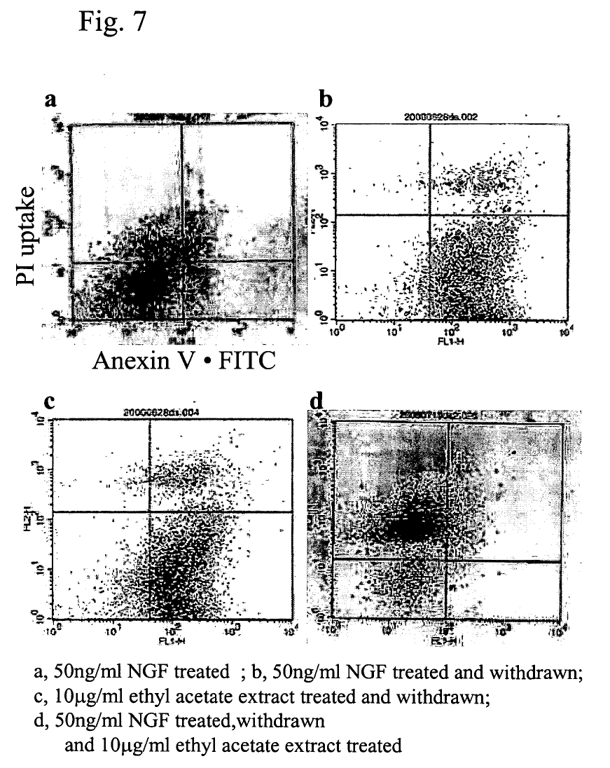

4 hours later when ethyl acetate soluble extract or NGF was removed, cells started the programmed cell death and 12 hours later, the number of cells in apoptosis was the largest (FIG. 7a, FIG. 7b, FIG. 7c and FIG. 7d).

### Experimental Example 7

### Gene Expression of NGF

7-1. Gene Expression Assay Using RT-PCT Method.

Total RNA of cell was extracted by Trizol B reagent (Gibco BRL Co.). Cells lysed by adding 1 ml of Trizol B were harvested by cell scraper. Cell suspension was transferred to 1.5 ml of microtube and pipetted several times to disrupt cells using syringe with 21G needle. Cell lysate was centrifuged at 12,000 rpm for 10 min at 4° C. The supernatant was mixed with chloroform and the mixture was shaked vigorously followed by incubation for 15 mins at room temperature. The mixture was centrifuged again at 12,000 rpm for 15 mins at 4° C. Supernatant containing RNA was transferred to a fresh microtube and it was mixed with 100% isopropanol by repeated inversion. The sample was allowed to incubation at room temperature and centrifuged at 12,000 rpm for 15 mins at 4° C. RNA pellet obtained by removing supernatant was washed with 1.0 ml of 75% ethanol and centrifuged at 12,000 rpm for 5 mins at 4° C. After complete dry of pellet at R.T., pellet was resuspended in DEPC (diethylpyrocarbonate)-treated water and UV absorbance was measured at 260 nm and 240 nm by using spectrophotometer (Bio-Rad, U.S.A.).

Complementary DNA (cDNA) was synthesized by reverse transcriptase and polymerase chain reaction was carried out by Taq polymerase (Takara Co., Japan).

1 μg of above prepared RNA was heat-treated at 65° C. for 15 mins to separate the RNA strand. Reaction reagent (4 μl of 5× reaction buffer, 1 μl of 10 mM dNTP mixture, 1 μl of 20 μM oligo(dT)₁₅ primer, 0.2 μl of M-MLV reverse transcriptase (200 U/μl), 2 μl of 0.1M dithiothreitol, DEPC-treated water up to 20 μl) was added to RNA sample, incubated at 37° C. for 60 mins and subsequently incubated again at 72° C. for 15 mins to synthesize the complementary DNA.

For polymerase chain reaction, 1 μl of above prepared cDNA was mixed with reaction solution (2 μl of 10× reaction buffer, 2 μl of 2.5 mM dNTP mixture, 0.2 μl of Taq polymerase (5 U/μl), 1 μl of 10 μM sense primer, 1 μl of 10 μM anti-sense primer, DEPC-treated water up to 25 μl) and performed for 30 cycles of PCR using thermocycler (Perkin Elmer Co.) as following condition disclosed in Table 3.

TABLE 3

| STEP | Temperature | Time | Cycle |
|---|---|---|---|
| Pre-denaturation | 95° C. | 3 min | 1 |
| Denaturation | 95° C. | 30 sec | 30 |
| Annealing | 62° C. | 30 sec | |
| Extension | 72° C. | 1 min | |
| Final-extension | 72° C. | 7 min | 1 |

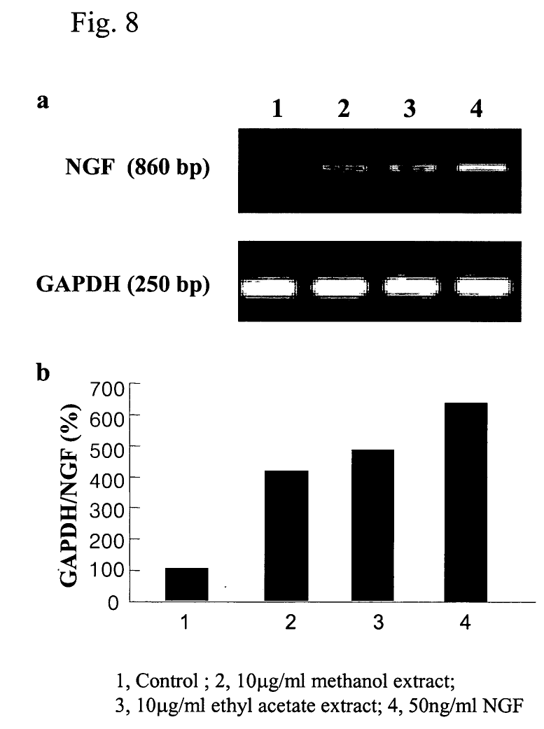

10 μl of RT-PCR product was loaded on 1.5% agarose (Gibco BRL Co., U.S.A.) gel and electrophoresis analysis thereofwas performed in 1×TAE buffer at 100 volts for 30 mins. The agarose gel was stained in ethidium bromide solution for 20 mins and destained in distilled water for 10 mins. The DNA bands of the gel were observed on the UV light box and then the gel was photographed by using Gel-DOC (Bio-Rad Co.)(FIG. 8a), which presented the evaluated result of electrophoresis (FIG. 8b).

250 bp of GAPDH was used as a standard for comparing NGF gene expression of each group, i.e. untreated control group, 50 ng/ml NGF treated group, 10 μg/ml the crude extract treated group and 10 μg/ml the ethyl acetate soluble extract treated group.

As shown in FIG. 8a and FIG. 8b, it was confirmed that the inventive extract increased the NGF receptor expression.

7-2. Gene Expression Assay Using Immunocytochemical Method.

To verify the effect of inventive extract on the expression of p75 receptor as NGF receptor, the immunocytochemical method was used in this experiment.

PC12 cell was grown in complete media on glass coverslip (22 mm×22 mm). 24 hours later, cultured cells were treated with 10 μg/ml of ethyl acetate soluble extract or 10 ng/ml of NGF, respectively. Another 48 hours later, cells were fixed with 2% paraformaldehyde dissolved in phosphate buffer (pH 7.4) for 30 mins at R. T. and then washed with PBS. The solution containing 2% BSA and 0.1% Triton X-100 was dropped on the cell and the coverslip kept on ice for 30 mins to remove the non-specific reaction and increase cell membrane permeability. The cells were rinsed with mixture of PBS and 1% BSA for 10 mins 3 times and primary antibody anti-p75 (1:2000 dilution, Chemicon International Co.) was added thereon and incubated for 1 hour.

Subsequently, cells were rinsed with mixture of PBS and 1% BSA for 10 mins 3 times and Texas Red conjugated-secondary antibody anti-mouse IgG (1:5000 dilution, WithLab Co.) was added thereon to incubate for another 1 hour. After reaction, the secondary antibody was removed and washed with PBS.

The result of receptor expression was observed by using fluorescence microscope.

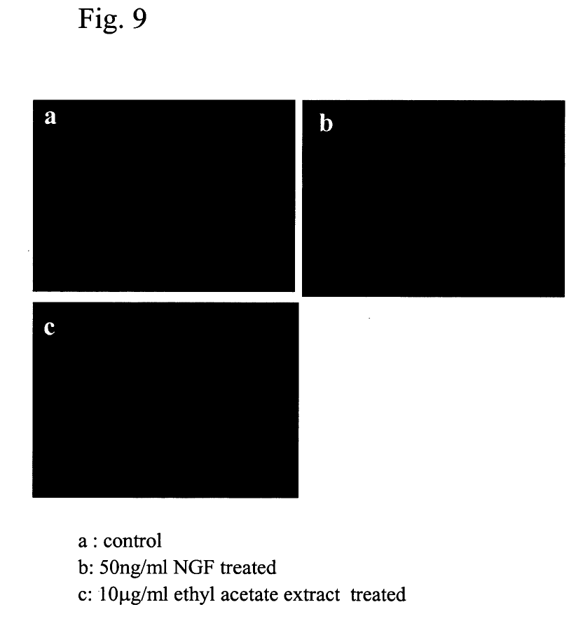

In the NGF treated group and inventive ethyl acetate soluble extract treated group, there was the increase of the p75 receptor expression comparing with that of control group (FIG. 9a, FIG. 9b and FIG. 9c).

It is confirmed that the inventive extract causes the increase the NGF synthesis, which leads the receptor expression to be augmented, and thereby it can be deduced that inventive extract induces the growth and differentiation of PC12 cells. Also, it could be thought that the NGF gene expression was stimulated by direct interaction of ethyl acetate soluble extract with receptor.

### Experimental Example 8

### Passive Avoidance Test

For passive avoidance test, ethyl acetate soluble extract was administered to the mice prior to scopolamine-induced amnesia.

An automated system with a shuttle box was used to evaluate the effects of the extracts on learning and memory associated with neuronal cell growth.

Avoidance shuttle box (40×20×20 cm, Gemini Co., U.S.A.) was divided into two chambers of equal size and had the 3 mm thickness of grid in interval of 0.5 cm on the floor of the box. A light chamber is equipped with an illuminator. Male mice weighing 25-30 g were initially placed in the light chamber.

Scopolamine was injected intraperitoneally to the mouse. 30 minutes after, acquisition training was carried out, which delivering the electrical foot shock (1 mA/10 g body weight) to the mouse through the grid floor when the mouse preferring darkness went out from light chamber and entered the dark chamber.

At 24 hours after the acquisition trial, the identical experiment was performed again with mouse to measure the latency time staying at the light chamber. The data was regarded as the index which meant the memory on previous training by electronic shock. Latency to enter the dark compartment was measured for 180 sec. If it did not enter the dark chamber within the cut-off time (180 sec), it was assigned a value of 180 sec as its latency.

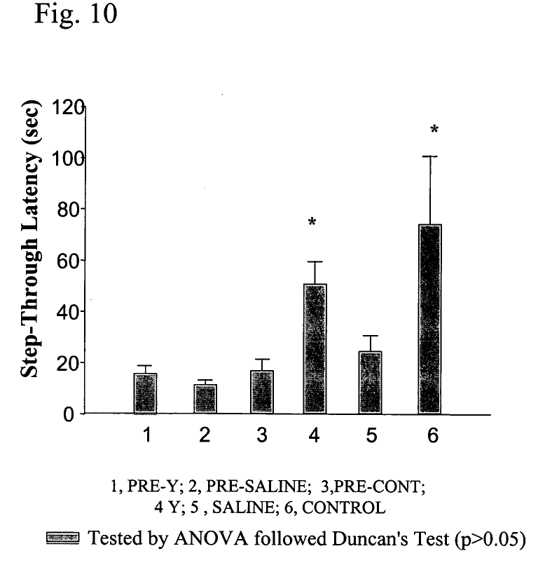

As shown in FIG. 10, the latency time was the shortest in scopolamine-induced amnesia mouse. The latency of ethyl acetate soluble extract treated mouse was increased.

The results indicate that inventive extract may improve memory and recognition ability and enhance passive recognition function by increasing NGF expression.

It was confirmed that the inventive Cistanche deserticola extract can be useful as an effective anti-dementia drug.

### Experimental Example 9

### Toxicity Test

Methods (1)

The acute toxicity tests on ICR mice (mean body weight 25±5 g) and Sprague-Dawley rats (235±10 g, Jung-Ang Lab Animal Inc.) were performed using the extract of the Example 1. Four group consisting of 10 mice or rats was administrated orally intraperitoneally with 250 mg/kg, 500 mg/kg, 1000 mg/kg and 5000 mg/kg of test sample or solvents (0.2 ml, i.p.) respectively and observed for 2 weeks.

Methods (2)

The acute toxicity tests on ICR mice and Sprague-Dawley rats were performed using the extract of the Example 1. Four group consisting of 10 mice or rats was administrated intraperitoneally with 25 mg/kg, 250 mg/kg, 500 mg/kg and 725 mg/kg of test sample or solvents (0.2 ml, i.p.), respectively and observed for 24 hours.

Results

There were no treatment-related effects on mortality, clinical signs, body weight changes and gross findings in any group or either gender. These results suggested that the extract prepared in the present invention were potent and safe.

Hereinafter, the formulating methods and kinds of excipients will be described, but the present invention is not limited to them. The representative preparation examples were described as follows.

Preparation of Powder

| | |
|---|---|
| Dried powder of Example 2-1 | 50 mg |
| Lactose | 100 mg |
| Talc | 10 mg |

Powder preparation was prepared by mixing above components and filling sealed package.

Preparation of Tablet

| | |
|---|---|
| Dried powder of Example 2-1 | 50 mg |
| Corn Starch | 100 mg |
| Lactose | 100 mg |
| Magnesium Stearate | 2 mg |

Tablet preparation was prepared by mixing above components and entabletting.

Preparation of Capsule

| | |
|---|---|
| Dried powder of Example 2-1 | 50 mg |
| Corn starch | 100 mg |
| Lactose | 100 mg |
| Magnesium Stearate | 2 mg |

Tablet preparation was prepared by mixing above components and filling gelatin capsule by conventional gelatin preparation method.

Preparation of Injection

| | |
|---|---|
| Dried powder of Example 2-1 | 50 mg |
| Distilled water for injection | optimum amount |
| PH controller | optimum amount |

Injection preparation was prepared by dissolving active component, controlling pH to about 7.5 and then filling all the components in 2 ml ample and sterilizing by conventional injection preparation method.

Preparation of Liquid

| Dried powder of Example 2-1 | 0.1˜80 g |
|---|---|
| Sugar | 5˜10 g |
| Citric acid | 0.05˜0.3% |
| Caramel | 0.005˜0.02% |
| Vitamin C | 0.1˜1% |
| Distilled water | 79˜94% |
| CO₂ gas | 0.5˜0.82% |

Liquid preparation was prepared by dissolving active component, filling all the components and sterilizing by conventional liquid preparation method.

Preparation of Health Food

| Dried powder of Example 2-1 | 1000 | mg |
|---|---|---|
| Vitamin mixture | optimum amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Amide nicotinic acid | 1.7 | mg |
| Folic acid | 50 | μg |
| Calcium pantothenic acid | 0.5 | mg |
| Mineral mixture | optimum amount | |
| Ferrous sulfate | 1.75 | mg |
| Zinc oxide | 0.82 | mg |
| Magnesium carbonate | 25.3 | mg |
| Monopotassium phosphate | 15 | mg |
| Dicalcium phosphate | 55 | mg |
| Potassium citrate | 90 | mg |
| Calcium carbonate | 100 | mg |
| Magnesium chloride | 24.8 | mg |

The above-mentioned vitamin and mineral mixture may be varied in may ways. Such variations are not to be regarded as a departure from the spirit and scope of the present invention.

Preparation of Health Beverage

| | |
|---|---|
| Dried powder of Example 2-1 | 1000 mg |
| Citric acid | 1000 mg |
| Oligosaccharide | 100 g |
| Apricot concentration | 2 g |
| Taurine | 1 g |
| Distilled water | 900 ml |

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

1. A pharmaceutical composition comprising the crude extract of Cistanche deserticola Y.C. MA as an active ingredient for the treatment and prevention of degenerative brain disease.

2. The pharmaceutical composition according to claim 1 wherein said crude extract is extracted with the solvent selected from the group consisting of water, lower alcohol and the mixture thereof.

3. The pharmaceutical composition according to claim 2 wherein said lower alcohol is methanol.

4. A pharmaceutical composition comprising the polar solvent soluble extract of Cistanche deserticola Y.C. MA as an active ingredient for the treatment and prevention of degenerative brain disease.

5. The pharmaceutical composition according to claim 4 wherein said polar solvent soluble extract is extracted with the solvent selected from the group consisting of water, lower alcohol and the mixture thereof.

6. The pharmaceutical composition according to claim 5 wherein said lower alcohol is butanol.

7. A pharmaceutical composition comprising the non-polar solvent soluble extract of Cistanche deserticola Y.C. MA as an active ingredient for the treatment and prevention of degenerative brain disease.

8. The pharmaceutical composition according to claim 7 wherein said non-polar solvent is selected at least one from the group consisting of hexane, carbon tetrachloride, chloroform, methylene chloride, ethyl ether and ethyl acetate.

9. The pharmaceutical composition according to claim 8 wherein said nonpolar solvent is ethyl acetate.

10. The pharmaceutical composition according to claim 1 wherein said extract is extracted from the plant consisting of Cistanche deserticola, C. salsa, C. ambigua or and the same genus plant.

11. The pharmaceutical composition according to claim 1 wherein said degenerative brain disease comprises stroke, Alzheimer's disease (AD), Parkinson's disease (PD), senile dementia.

12. The pharmaceutical composition according to claim 1 wherein said composition is containing the extract between 0.1 to 50% by weight based on the total weight of the composition.

13. A use of a crude extract, polar solvent soluble or non-polar solvent soluble extract of Cistanche deserticola Y.C. MA for the preparation of therapeutic agent for the treatment and prevention of degenerative brain disease.

14. A health food comprising the crude extract of Cistanche deserticola Y.C. MA, together with a sitologically acceptable additive for the prevention and improvement of degenerative brain disease.

15. The health food according to claim 14 wherein said health food is provided as powder, granule, tablet, chewing tablet, capsule or beverage type.
